(19) United States
(12) Patent Application Publication
(10) Pub. No.: US 2008/0009743 A1
(43) Pub. Date: Jan. 10, 2008

(54) ULTRASOUND DIAGNOSIS APPARATUS, ULTRASOUND PROBE AND BIOPSY NEEDLE ATTACHMENT

(76) Inventor: Kazuyoshi Hayasaka, Tokyo (JP)

Correspondence Address: Patrick W. Rasche;Armstrong Teasdale LLP, Suite 2600, One Metropolitan Square, St. Louis, MO, 63102, US

(21) Appl. No.: 11/760,248

(22) Filed: Jun. 8, 2007

(30) Foreign Application Priority Data
Jun. 15, 2006 (JP) 2006-165758

(51) Int. Cl. A61B 8/00; A61B 17/34; A61B 8/14

(52) U.S. Cl. 600/461

(57) ABSTRACT

To confirm the proper attachment of a biopsy needle attachment and to detect the type of the attached attachment, the body unit determines the proper attachment of the attachment if all bar-code readers of the ultrasound probe reads successfully the bar-code of the attachment at the opposing position, to display a guideline corresponding to the type of the attachment on the display unit, otherwise the body unit determines improper attachment of the attachment to the ultrasound probe and warns of the improper attachment.

### BACKGROUND OF THE INVENTION

The present invention relates to an ultrasound diagnosis apparatus, ultrasound probe, and biopsy needle attachment, which allow a biopsy (pathohistological tissue diagnosis) to be conducted during an ultrasound diagnosis.

An ultrasound diagnosis apparatus transmits ultrasound waves into an object (lesion) and receives echoes therefrom to generate a tomographic image of the lesion and a variety of diagnosis information based on the received echo signals.

To conduct a biopsy of a lesion, a centesis by means of a biopsy needle is conducted at the same time to the ultrasound diagnosis. For puncturing a biopsy needle, an attachment (guide tool) for biopsy is attached to the ultrasound probe that transmits ultrasound waves, then a biopsy needle is attached to the attachment. The attachment may have a plurality of guide holes for example and the user may select an appropriate one guide hole among a plurality of guide holes to insert the biopsy needle to be used for the puncture.

In the conventional manner, when an attachment for the biopsy needle is attached to the ultrasound probe, the attachment must be properly attached to the probe and positively secured to it.

However there have been cases in which the attachment may have a difficulty to fixedly secure to the probe due to the shape of the attachment. In such a case, disadvantageously, to determine whether the attachment is securely mounted to the probe or not, the user must visually verify the attaching site of the attachment.

To remedy the disadvantage as have been described above there has been proposed a technique as disclosed in the following patent reference 1 for example.

[patent reference 1] Japanese Unexamined Patent Publication No. 2006-122490

In the patent reference 1 above, there is disclosed an ultrasound diagnosing system in which a probe identification hole is provided to the ultrasound probe at a different position by the type of the probe, a photo-sensitive switch just beneath the hole is also provided, and the biopsy adaptor (biopsy needle attachment), which fits to the ultrasound probe, is provided with a shielding projection at the position of blocking the light path of the photo-sensitive switch by inserting into the probe identification hole at the time when properly attached to the ultrasound probe, so as to identify the accommodation of the ultrasound probe with the biopsy needle adaptor automatically by the ultrasound apparatus to display light or wrong state of fit in a display unit.

In accordance with the ultrasound diagnosis apparatus disclosed in the patent reference 1, the puncture is prevented from being performed in a wrong state in which a biopsy needle adaptor is attached to a wrong ultrasound probe because it has an identical shape or similar shape to the right ultrasound probe, or is prevented from being performed in an improper attachment between a right biopsy needle adaptor and the ultrasonic probe.

However in the ultrasound diagnosis apparatus disclosed in the patent reference 1, each ultrasound probe has only one single photo-sensitive switch and each biopsy needle adaptor has only one single shielding projection, and the attachment is detected to be fit with the probe once the shielding projection blocks the photo-sensitive switch, therefore there is a disadvantage that it cannot detect the case in which, although the shielding projection covers the photo-sensitive switch, the biopsy needle adaptor is obliquely attached to the ultrasound probe and so the biopsy needle adaptor is not completely fit with the ultrasound probe.

In addition, there is another disadvantage that, if there are many types of biopsy needle adaptors for only one ultrasound probe, since the ultrasound diagnosis apparatus disclosed in the patent reference 1 may not detect the type of the adaptor, the guideline displayed on the display unit may not be the guideline fit to the biopsy needle adaptor.

### SUMMARY OF THE INVENTION

Therefore, an object of the present invention is to provide an ultrasound diagnosis apparatus, an ultrasound probe and a biopsy needle attachment which may detect whether the biopsy needle attachment is properly fit to the probe and which may also detect the type of the attachment attached thereto.

In order to achieve the object listed above, an ultrasound diagnosis apparatus for biopsy in accordance with first aspect, is an ultrasound diagnosis apparatus for biopsy comprising an ultrasound probe for attaching a biopsy needle attachment for securing a biopsy needle, in which the ultrasound probe includes a plurality of detector devices for detecting whether the biopsy needle attachment is properly attached thereto, at least one of the plurality of detector devices is a recorder unit reader, the biopsy needle attachment has a recording unit for storing the information about the biopsy needle attachment at the position opposing to the recorder unit reader when attached properly to the ultrasound probe, the recording unit reader reads out the information about the biopsy needle attachment from the recorder unit, and if all of the detecting devices detects the proper attachment of the biopsy needle attachment to the ultrasound probe, the apparatus displays a guideline appropriate for the biopsy needle attachment being attached based on the information about the biopsy needle attachment read out by the recording unit reader, otherwise the apparatus warns of improper attachment of the biopsy needle attachment to the ultrasound probe.

The ultrasound probe in accordance with a second aspect is an ultrasound probe of an ultrasound diagnosis apparatus for biopsy for attaching a biopsy needle attachment for securing a biopsy needle, comprising a plurality of detector devices for detecting whether the biopsy needle attachment is properly attached, in which at least one of the plurality of detector devices is a recorder unit reader.

The biopsy needle attachment in accordance with a third aspect of the present invention is a biopsy needle attachment for attaching to an ultrasound probe of an ultrasound diagnosis apparatus for biopsy, comprising a recorder unit for storing the information about the biopsy needle attachment at the position opposing to the recorder unit reader of the ultrasound probe when properly attached to the ultrasound probe.

In accordance with the present invention, an ultrasound diagnosis apparatus, an ultrasound probe and a biopsy needle attachment are provided which allow the user to confirm whether the biopsy needle attachment is properly attached, and to detect the type of attachment being attached.

Further objects and advantages of the present invention will be apparent from the following description of the preferred embodiments of the invention as illustrated in the accompanying drawings.

### BRIEF DESCRIPTION OF THE DRAWINGS

FIG. 1 shows a schematic block diagram of an ultrasound diagnosis apparatus 1000 in accordance with the preferred embodiment of the present invention;

FIG. 2 shows a side view of an attachment 300;

FIG. 3 shows a plan view of a attachment 300; and

FIG. 4 shows a flow chart illustrating the operation of the ultrasound diagnosis apparatus 1000 in accordance with the preferred embodiment of the present invention at the time of puncture.

### DETAILED DESCRIPTION OF THE INVENTION

Now the ultrasound diagnosis apparatus 1000 in accordance with the preferred embodiment will be described in greater details herein below.

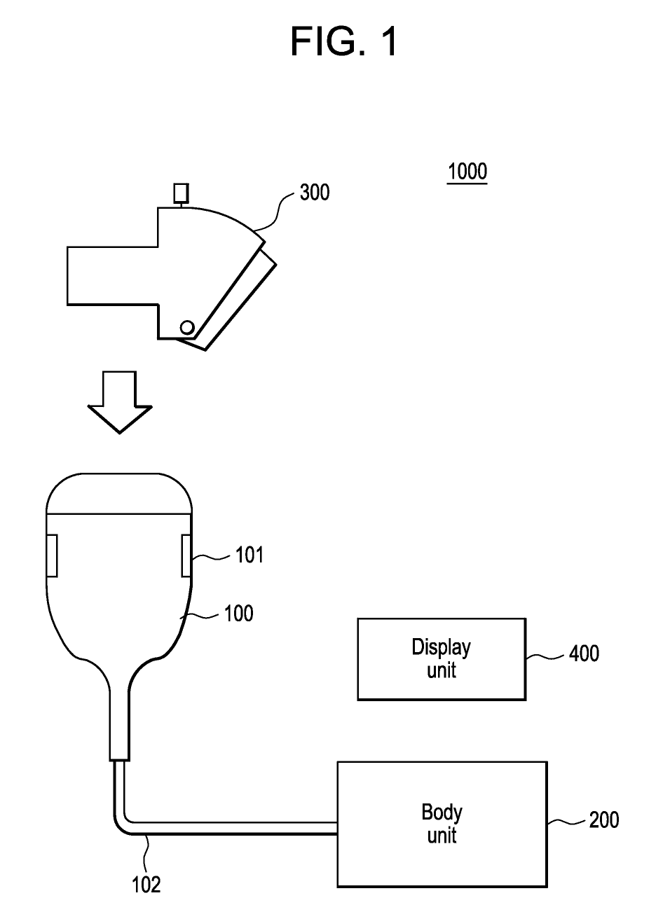

Referring to FIG. 1, there is shown a schematic block diagram of the ultrasound diagnosis apparatus 1000 in accordance with the preferred embodiment.

As shown in FIG. 1, the ultrasound diagnosis apparatus 1000 has an ultrasound probe 100, a body unit 200, and a display unit 400, and an attachment 300 for attaching a biopsy needle is attached to the ultrasound probe 100.

The ultrasound probe 100 is used by a user such as a doctor to place in contact with an examinee.

The ultrasound probe 100 has a bar-code reader 101 on one side at the position opposite to a bar-code 302 of the attachment 300 as will be described later.

The bar-code reader 101 reads the bar-code 302 as will be described later.

The ultrasound probe 100 is connected to the body unit 200 by a signal cable 102.

The body unit 200 supplies driving signals to the ultrasound probe 100 to transmit ultrasound.

The body unit 200 receives echo signals received by the ultrasound probe 100, and generates a tomographic image based on the signals to display on the display unit 400.

The body unit 200 determines whether or not the attachment 300 is properly connected to the ultrasound probe 100 based on the signal received from the bar-code reader 101. The decision may be made based on the status for example whether the bar-code reader 101 reads out the bar-code correctly or not.

Here the expression that the attachment 300 is correctly attached to the ultrasound probe 100 means the status that the attachment 300 is completely secured to the ultrasound probe 100, and the attachment 300 does not slip with respect to the ultrasound probe 100 even when performing the operation on the attachment 300 such as changing the puncture angle of the biopsy needle, as will be described later.

The body unit 200 determines the type of the attachment 300 (such as that for a linear probe, for a convex probe, or the angle of biopsy needle) based on the signals received from the bar-code reader 101.

Once the attachment 300 is confirmed to be properly attached and the type thereof is identified, the puncture path of the biopsy needle in correspondence with the type of the attachment 300 identified will be displayed as the guideline on the tomographic image. The guideline is an image indicating the puncture path of the biopsy needle within the examinee when puncturing by means of the attachment 300. For example, the types of the attachment 300 and the guideline information in correspondence with each of the types may be stored on a storage unit not shown in the figure and any necessary information may be read out therefrom to display overlapped on a tomographic image.

The body unit 200 may display a warning display indicating “attachment not properly attached” instead of the guideline if the attachment 300 is determined not to be properly attached.

More specifically, when the ultrasound probe 100 is connected to the body unit 200 with the signal cable 102 and the power is supplied to the body unit 200, and once the function of displaying the puncture guideline is selected, the guideline will be displayed if the attachment 300 is properly attached, otherwise a warning display will be displayed on the display unit 400.

Although in the above description a warning display is used to warn the user if the attachment 300 is not properly attached, an alarm such as a buzzer in the body unit 200 may be provided to inform the user by the alert sound instead of displaying a warning sign.

The display unit 400 is connected to the body unit 200 to display the tomographic image and guideline generated by the body unit 200.

To the ultrasound probe 100, an attachment 300 for securing a biopsy needle is attached. A typical example of the attachment will be described herein below.

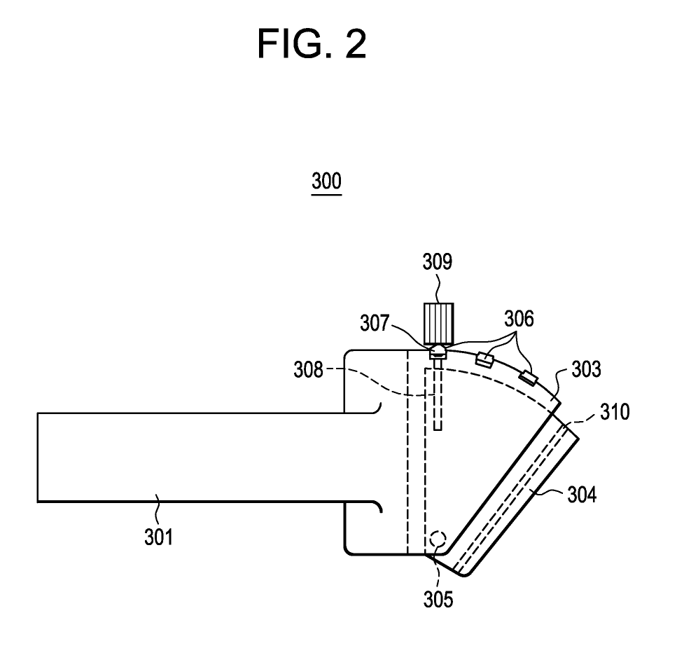

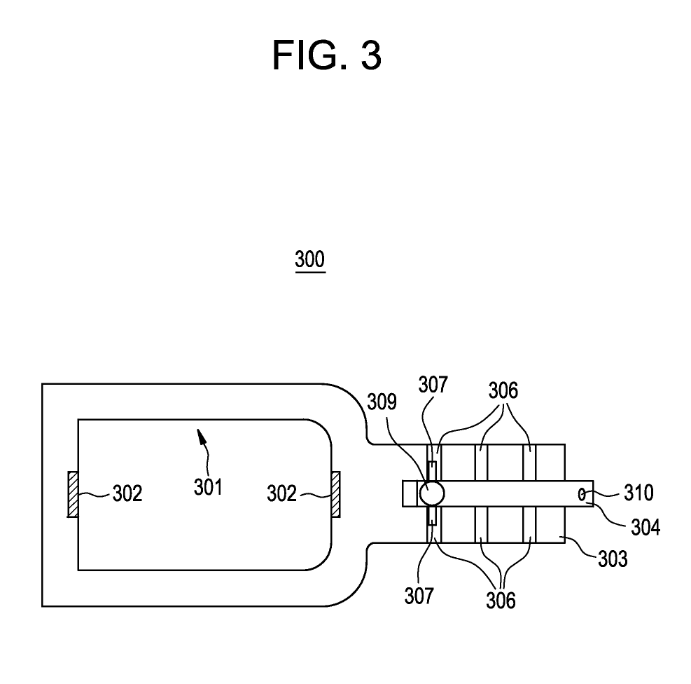

Referring to FIG. 2 and FIG. 3 there is shown a schematic diagram of the attachment 300.

FIG. 2 shows a side view of the attachment 300, and FIG. 3 shows a plan view of the attachment 300.

As shown in FIG. 2, the attachment 300 has a probe connector 301, a bar-code 302, a fixed part 303, a movable part 304, an axis 305, a groove 306, a side pin 307, a vertical pin 308, a knob 309, and a guide hole 310.

The probe connector 301 is for connecting to the ultrasound probe 100, and has an annular ring structure with the inner diameter engageable to the outer diameter of the ultrasound probe 100.

The bar-code 302 is placed inside the annular ring of the probe connector 301, at the position of contacting to the ultrasound probe 100. The bar-code 302 is placed at the position opposed to the bar-code reader 101 of the ultrasound probe 100 as will be described later when the attachment 300 is properly attached to the ultrasound probe 100. In this preferred embodiment the two bar-codes 302 will be described to be placed at positions opposite to each other on opposite sides of the annular ring of the probe connector 301.

In the bar-code 302 the information about the attachment 300 is printed, if the attachment 300 is properly attached to the ultrasound probe 100 then the information will be read out by the bar-code reader 101.

The information about the attachment 300 is the information on the type of the attachment 300 and the like. The same information is printed on the every bar-code 302.

The fixed part 303 is generally U-shaped plate member, and the movable part 304 is a generally fan-shaped plate member. The movable part 304 is sandwiched by the fixed part 303 from both sides.

The movable part 304 is swingable with respect to the fixed part 303 about the axis 305 placed at the point corresponding to the center of the fan. The swing angle of the movable part 304 is defined by the groove 306 formed in the fixed part 303 and the side pin 307 which fits into the groove. The groove 306 is provided along with the direction of thickness of the plate at the edge of the side opposite to the axis 305 of the fixed part 303. In the example shown in FIG. 2 and FIG. 3 there are three grooves 306 at a predetermined interval. Three grooves illustrated is merely an example and the number of grooves may be any number more than one.

The side pin 307 is placed perpendicular to the vertical pin 308. The vertical pin 308 is implanted on the side opposite to the axis 305 of the movable part 304. The vertical pin 308 has the knob 309 at one end, and the vertical pin 308 is inserted or withdrawn by moving up and down the knob 309. More specifically, by releasing the engagement of the groove 306 with the side pin 307 by pulling the vertical pin 308, the movable part 304 becomes rotatable, and by engaging the side pin 307 to another groove 306 by rotating the movable part 304, the swing angle of the movable part 304 may be altered.

The movable part 304 has the guide hole 310 for the biopsy needle on the end of the fan opposite to the end having the vertical pin 308 implanted. The guide hole 310 is inserted through the movable part 304 radially in the fan. A biopsy needle is inserted through the guide hole 310. The direction of the biopsy needle is defined by the angle of the center axis of the guide hole 310. The angle of the center axis of the guide hole 310, that is, the puncture angle of the biopsy needle, may be altered by changing the swing angle of the movable part 304.

The operation of the ultrasound diagnosis apparatus 1000 at the time of biopsy needle puncture in accordance with the preferred embodiment will be described in greater details.

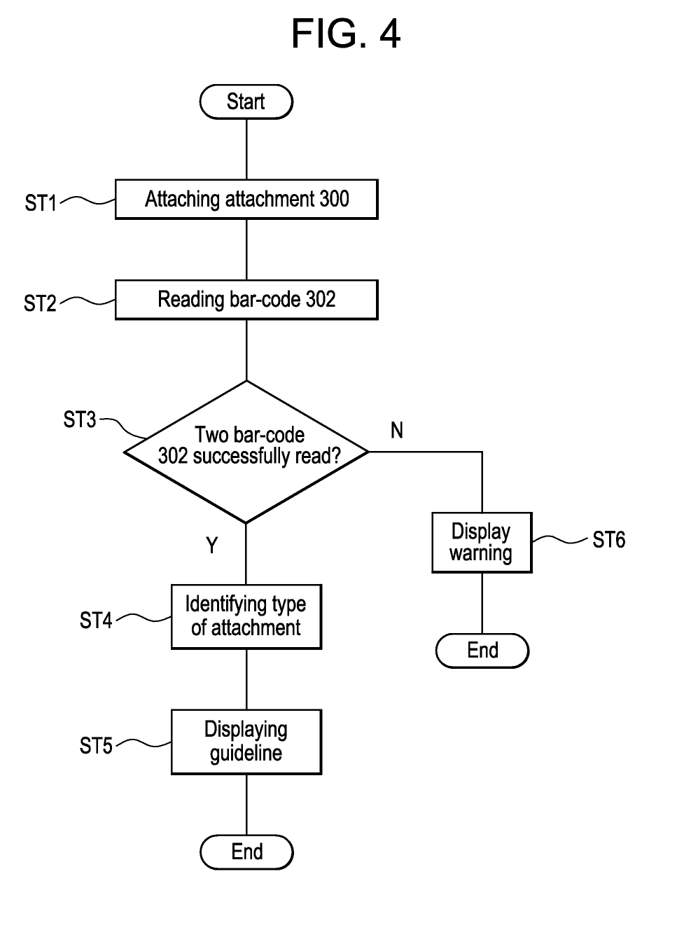

Referring to FIG. 4 there is shown a flow chart for the exemplary operation of the ultrasound diagnosis apparatus 1000 in accordance with the preferred embodiment at the time of biopsy needle puncture.

Step ST1:

The attachment 300 is attached to the ultrasound probe 100.

Step ST2:

The bar-code reader 101 reads the bar-code 302.

Step ST3:

In step ST2 if both of two bar-code readers 101 are successfully reading the respective bar-codes 302, then the attachment 300 is determined to be properly attached, and the process proceeds to step ST4. If either or none of the two bar-code readers 101 fails to read the bar-code 302, then the attachment 300 is determined not to be properly attached, and the process proceeds to step ST6.

Step ST4:

The body unit 200 determines the type of the attachment 300 attached in step ST1 based on the information about the attachment 300 read from the bar-code 302 by the bar-code reader 101 in step ST2.

Step ST5:

The body unit 200 displays on the display unit 400 a guideline appropriate for the type of the attachment 300 determined in step ST4.

As can be appreciated from the foregoing description, in accordance with the ultrasound diagnosis apparatus 1000 of the preferred embodiment, the attachment 300 has two bar-codes 302, and when the attachment 300 is attached to the ultrasound probe 100, the same number of bar-code readers 101 as the bar-codes 302 are provided on the ultrasound probe 100 at the position opposite to the bar-code 302 when properly attached. In this manner if every bar-code reader 101 reads out successfully the bar-code 302 at the opposite position, then the body unit 200 determines that the attachment 300 is properly attached to the ultrasound probe 100. If either one of them is not successfully read out, then the body unit 200 determines that the attachment 300 is not properly attached to the ultrasound probe 100. Furthermore, when the body unit 200 determines that the attachment 300 is properly attached to the ultrasound probe 100 then it displays a guideline appropriate for the type of the attachment 300 on the display unit 400 based on the information on the read bar-code 302, whereas if the body unit 200 determines the attachment 300 is not properly attached to the ultrasound probe 100 then it warns of the improper attachment by means of a warning display.

It should be noted that the present invention is not limited to the preferred embodiment described above.

More specifically, any changes, combination, sub-combination, replacement of the components of the preferred embodiment described above may be made when implementing the present invention, within the technical scope or the equivalent of the present invention.

Although in the preferred embodiment as have been described above, the attachment 300 has two bar-codes 302 and the ultrasound probe 100 has bar-code readers 101 to read them, the present invention is not limited thereto. For example, the attachment 300 may also have two or more storage units such as IC tags or magnetic cards, and the ultrasound probe 100 may have readers for the storage units at the opposite positions. These storage units store the information about the attachment 300.

In addition, the ultrasound probe 100 may have two or more noncontact sensors by means of which it can detect the loading of the attachment 300, or the attachment 300 has projections and the ultrasound probe 100 has physical switches at the opposite positions thereto to enable detecting the loading of the attachment 300. However, because in these alternative examples, the body unit 200 may not be able to determine the type of the attachment 300 to be connected, the attachment 300 should at least one bar-code 302 or storage unit, and the noncontact sensors or physical switches in addition thereto.

In summary, the present invention is applied to an ultrasound diagnosis apparatus, an ultrasound probe, and an attachment, which may have two or more detection mechanisms constituted of the detected unit in the attachment side and the detecting unit in the ultrasound probe, such as the bar-code and the bar-code reader, IC tag and IC tag reader, noncontact sensor, the projection and physical switches and the like, in which the attachment is determined to be properly mounted to the ultrasound probe by detecting the detected units by all of the detecting units, and at least one of the two or more detecting mechanisms may be constituted of a storage unit for storing the information about the attachment and the storage unit reader.

Although in the preferred embodiment as have been described above, the information stored in the bar-code 302 about the attachment 300 has been described to be the information about the type of the attachment 300, the information may further include the information about the puncture angle of the biopsy needle if the attachment is not capable of changing the puncture angle of the biopsy needle.

Many widely different embodiments of the invention may be configured without departing from the spirit and the scope of the present invention. It should be understood that the present invention is not limited to the specific embodiments described in the specification, except as defined in the appended claims.

1. An ultrasound diagnosis apparatus for use in a biopsy, having an ultrasound probe for attaching a biopsy needle attachment for securing the biopsy needle, wherein:
the ultrasound probe has a plurality of detection devices for detecting the proper attachment of the biopsy needle attachment;
at least one of the plurality of detection devices is a recording unit reader;
the biopsy needle attachment has a recording unit for storing the information about the biopsy needle attachment at the position opposite to the recording unit reader when properly attached to the ultrasound probe;
the recording unit reader reads out the information about the biopsy needle attachment from the recording unit; and
if all of the detecting devices detects the proper attachment of the biopsy needle attachment to the ultrasound probe, the apparatus displays a guideline appropriate for the biopsy needle attachment being attached based on the information about the biopsy needle attachment read out by the recording unit reader, otherwise the apparatus warns of improper attachment of the biopsy needle attachment of the ultrasound probe.

2. An ultrasound diagnosis apparatus according to claim 1, wherein:
the guideline is a line defining the boundary of insertion area of the biopsy needle.

3. An ultrasound diagnosis apparatus according to claim 1, wherein:
the information about the biopsy needle attachment includes the type of the biopsy needle attachment and the puncture angle of the biopsy needle.

4. An ultrasound diagnosis apparatus according to claim 1, wherein:
the body unit has a database for storing the guideline information for displaying the guideline on the tomographic image in correspondence with the type of the biopsy needle attachment.

5. An ultrasound diagnosis apparatus according to claim 1, further comprising:
a body unit for generating a tomographic image based on echo signals received by the ultrasound probe; and
a display unit for displaying the tomographic image generated by the body unit;
wherein:
the body unit displays on the display unit the guideline over the tomographic image if all of the detection devices detects the proper attachment of the biopsy needle attachment to the ultrasound probe.

6. An ultrasound diagnosis apparatus according to claim 1, wherein:
the recording unit is a bar-code; and
the recording unit reader is a bar-code reader.

7. An ultrasound diagnosis apparatus according to claim 1, wherein:
the recording unit is an IC tag; and
the recording unit reader is an IC tag reader.

8. An ultrasound diagnosis apparatus according to claim 1, wherein:
the recording unit is a magnetic card; and
the recording unit reader is a magnetic card reader.

9. An ultrasound probe used in an ultrasound diagnosis apparatus for biopsy for attaching a biopsy needle attachment securing a biopsy needle, comprising:
a plurality of detection devices for detecting the proper attachment of the biopsy needle attachment,
wherein:
at least one of the plurality of detection devices is a recording unit reader.

10. An ultrasound probe according to claim 9, wherein:
the recording unit reader reads out the information about the biopsy needle attachment recorded in a recording unit mounted on the biopsy needle attachment at the position opposed to the recording unit reader if the biopsy needle attachment is properly attached.

11. An biopsy needle attachment attached to an ultrasound probe of an ultrasound diagnosis apparatus for biopsy, comprising:
a recording unit for storing the information about the biopsy needle attachment at the position opposite to a recording unit reader mounted on the ultrasound probe if properly attached to the ultrasound probe.

12. An ultrasound diagnosis apparatus for use in a biopsy; said ultrasound diagnosis apparatus comprising:
an ultrasound probe comprising a bar code;
a biopsy needle attachment; and
a body unit comprising a bar code reader configured to read said bar code, said body unit is configured to determine if said biopsy needle attachment is coupled to said ultrasound probe based on a signal generated by said bar code reader.

13. An ultrasound diagnosis apparatus in accordance with claim 12, wherein said body unit is further configured to determine a type of said biopsy needle attachment based on the signal generated by said bar code reader.

14. An ultrasound diagnosis apparatus in accordance with claim 12, wherein said body unit is further configured to generate a visual indication if said biopsy needle attachment is not properly coupled to said ultrasound probe.

15. An ultrasound diagnosis apparatus in accordance with claim 12, wherein said body unit is further configured to generate an audio indication if said biopsy needle attachment is not properly coupled to said ultrasound probe.

16. An ultrasound diagnosis apparatus in accordance with claim 12, wherein said biopsy needle attachment comprises an annular ring that is configured to engage said ultrasound probe.

17. An ultrasound diagnosis apparatus in accordance with claim 16, wherein said bar code is disposed inside said annular ring.

18. An ultrasound diagnosis apparatus in accordance with claim 17, further comprising a second bar code is disposed inside said annular ring.

19. An ultrasound diagnosis apparatus in accordance with claim 12, wherein said bar code comprises information related to a type of said biopsy needle attachment.

20. An ultrasound diagnosis apparatus in accordance with claim 20, wherein said bar code comprises information related to a puncture angle of a biopsy needle coupled to said biopsy needle attachment.
